(19) United States
(12) Patent Application Publication
(10) Pub. No.: US 2026/0315793 A1
(43) Pub. Date: Oct. 15, 2026

(54) Electrical Stimulation Device for Assisting Digit Movement

(71) Applicant: Kiafar Haghkerdar, Chelsea, MA (US)

(72) Inventor: Kiafar Haghkerdar, Chelsea, MA (US)

(21) Appl. No.: 19/174,330

(22) Filed: Apr. 9, 2025

(51) Int. Cl. A61N 1/36

(52) U.S. Cl. CPC A61N 1/36003; A61N 1/36034

(57) ABSTRACT

This invention provides a wearable electrical stimulation device to assist the movement of a patient's digits. Worn on the forearm, it delivers targeted electrical pulses via electrodes, with additional manual or voice-triggered control. The device supports rehabilitation by facilitating digit flexion or extension, enhancing motor recovery in neurologically impaired individuals.

### FIELD OF THE INVENTION

This invention relates generally to electrical stimulation(ES) devices, and specifically to a wearable ES device for helping patients to relearn movement of the digits of an affected hand. Herein, we define “digits” to be fingers, or thumbs, or both fingers and thumbs. We define “movement” to be extension, or flexion, or both extension and flexion.

### BACKGROUND OF THE INVENTION

ES is a technique that uses electrical pulses to activate nerves and muscles in patients with neurological impairments, such as those resulting from, but not limited to, strokes. ES devices are well known in the art. Such devices include the XFT-2003EA hand rehabilitation device. Similar devices include the Saebo MyoTrac Infiniti hand rehabilitation device. ES devices receive signals from the patient's brain indicating that the patient intends to make a movement of the digits. ES devices typically comprise a plurality of electrodes connected to a control unit. At least one electrode of the plurality of electrodes delivers electrical pulses to targeted muscle groups. For hand rehabilitation in stroke patients, for example, ES devices generally require the plurality of electrodes to be placed on the forearm, targeting the flexor and extensor muscles.

Some patients recover the ability to flex their digits before they regain their ability to extend them. In contrast, some patients recover the ability to extend their digits before they regain their ability to flex them. Thus, for example, patients may be able to grasp an object by flexing their digits by volition but may have difficulty releasing the object by extending their digits by volition. Therefore, it is desirable to include a means for the patient using the ES device to stimulate the movement of the digits by other than pure volition. For example, the means to stimulate movement may include a button on the ES device that the patient can press with their unaffected hand to stimulate the movement of the digits of the affected hand. Herein, we define a “button” to be an electrical switch that operates when touched. The switch may be a physical one, or may be an electronic one that is touch sensitive, and may be represented, for example, as an icon on a touchscreen or the like. The means to stimulate movement may include a voice-activated control. In this case, the ES device may include a microphone and audio control circuitry configured to trigger digit movement pulses. Further, the voice-activated control may respond to specific words to stimulate movement of the patient's digits. The specific words may include words such as “extend” or “flex,” or the like in English or their equivalents in other languages.

### SUMMARY OF THE INVENTION

The present invention provides a wearable ES device to be positioned on the forearm, designed to aid in the rehabilitation of stroke or other patients by assisting in the movement of the digits. For example, for patients who can flex but not extend their digits by volition, the ES device provides a manual or voice-activated means to stimulate the digits to extend. Alternatively for example, for patients who can extend but not flex their digits by volition, the ES device provides a manual or voice-activated means to stimulate the digits to flex. The present invention provides an ES device for assisting digit movement rehabilitation in a patient, the ES device comprising: a control unit to be worn on a forearm of the patient, the control unit comprising: electronic circuitry configured to provide electrical pulses; an electrode connected to the electronic circuitry and positioned on the forearm such that the electrical pulses stimulate movement of the digits; and means for manually triggering the electrical pulses; and means for holding the control unit on the forearm. The means for manually triggering the electrical pulses can comprise a button positioned on the control unit. The means for manually triggering the electrical pulses can comprise a touch sensitive switch represented as an icon on a touchscreen of the control unit. The means for manually triggering the electrical pulses can comprise a voice-activated unit. The voice-activated unit may respond to specific words to stimulate extension or flexion of the patient's digits.

The ES device delivers targeted electrical pulses based on user-defined settings or automated algorithms that adapt stimulation patterns in response to biofeedback sensors detecting muscle activation levels. By facilitating controlled digit movements, the device helps stroke or other patients regain voluntary motor control more effectively. The means for manually triggering the movement of the digits allows the patient to grasp or release items more easily.

### BRIEF DESCRIPTION OF THE DRAWINGS

FIG. 1 shows a top view of a typical ES device according to the present invention.

FIG. 2 shows a side view of a typical ES device according to the present invention.

FIG. 3 shows an underneath view of a typical ES device according to the present invention.

### DETAILED DESCRIPTION OF THE INVENTION

The ES device comprises a means such as an adjustable band for holding a control unit equipped with integrated electrodes that are positioned to optimize stimulation of digit movement, ensuring precise and functional movement control. The control unit governs the delivery of electrical pulses, and allows for adjusting intensity, frequency, and duration based on programmed rehabilitation protocols. The ES device can further comprise wireless connectivity via Bluetooth or Wi-Fi to an external application, enabling therapists and patients to customize therapy sessions and track progress over time. The ES device can further comprise a touchscreen interface that allows patients to easily select stimulation modes, adjust intensity, and monitor progress. The ES device may connect wirelessly to a mobile device, allowing the ES device to be controlled through an application on the mobile device.

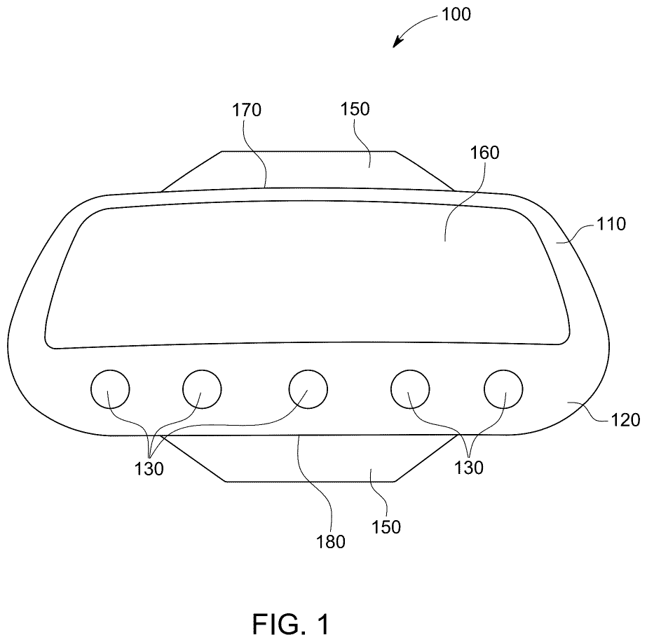

Referring to FIG. 1, ES device 100 comprises control unit 110 to be held against the patient's forearm with adjustable band 150. Control unit 110 includes multifunction control panel 120 with a plurality of buttons 130. Plurality of buttons 130 may be physical switches, touch-sensitive switches, capacitive sensors, or the like that allow the patient to perform basic operations with control unit 110 such as switching it off or on, starting a preprogrammed operation, increasing or decreasing intensity of pulses, or the like.

One or more buttons of the plurality of buttons 130 may be programmed specifically to stimulate movement of the patient's digits. This will help patients who have not yet regained the ability to extend or flex their digits. Specifically, some patients can grasp items by flexing their digits by volition, but may need the assistance of manual or voice-activated control of the ES device to extend their digits and thereby release the items. In contrast, some patients may need the assistance of manual or voice-activated control of the ES device to flex their digits to grasp items, but can extend their digits by volition to release the items.

ES device 100 may also include touchscreen 160 through which information regarding the operation of ES device 100 may be viewed, and by which more complicated commands to ES device 100 may be communicated. Touchscreen 160 may also include functionality for the patient to instruct ES device 100 to stimulate movement of the digits.

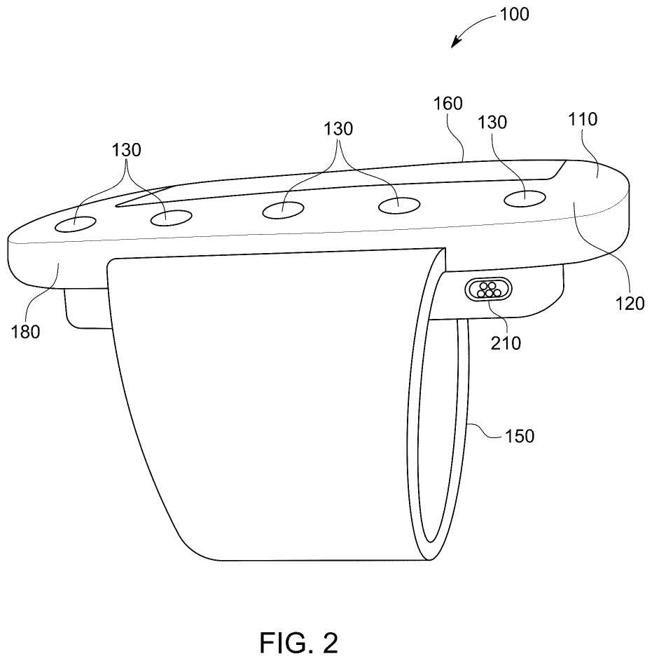

FIG. 2 shows a side view of ES device 100. In this view, charging port 210 is visible. Charging port 210 can be used to charge a rechargeable battery (not shown) in ES device 100. Adjustable band 150 may be a single continuous expandable or elastic strap with one end attached to side 170 and the other end attached to side 180. Alternatively, adjustable band 150 may comprise two straps, one connected to side 170 and one connected to side 180. The two straps comprise the adjustable band that may be fastened together by hook-and-eye fasteners, a buckle, or the like.

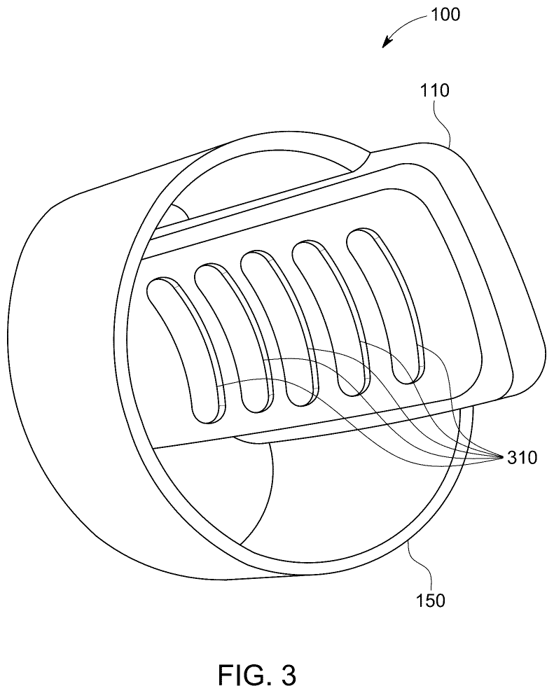

FIG. 3 shows an underneath view of ES device 100. Plurality of electrodes 310 are integrated into ES device 100. When ES device 100 is placed on the patient's forearm and band 150 is tightened, plurality of electrodes 310 are pressed against the patient's skin. One or more of plurality of electrodes 310 may be configured to receive signals from the patient's brain. One or more of plurality of electrodes 310 may be configured to stimulate movement of the patient's digits. This allows for the reception of electrical pulses from the patient's brain and the electrical stimulation of movement of the patient's digits.

The ES device can operate in various modes, including, but not limited to, the following.
Assisted Movement Mode: This mode stimulates muscles in a controlled sequence to facilitate digit movements.
Repetitive Training Mode: This mode repeats specific movement patterns to reinforce neuroplasticity.
Biofeedback-Responsive Mode: This mode adjusts stimulation intensity based on patient effort, promoting active participation in therapy.
Manual Override Mode: This mode allows the patient to manually trigger stimulation through an external control such as a button, touchscreen icon, or voice command, assisting in cases where voluntary movement is impaired.

In some embodiments, the ES device operates on a rechargeable battery. In some embodiments, the ES device provides adjustable pulse width (typically, but not exclusively, 20 to 300 μs), adjustable frequency (typically, but not exclusively, 1 to 100 Hz), or adjustable amplitude (typically, but not exclusively, 1 to 100 mA) for customized therapy. In some embodiments, the ES device comprises a touchscreen panel and mobile app integration for remote control and monitoring.

### Therapeutic Applications and Use Cases

The ES device is intended for, but not limited to, post-stroke, traumatic brain injury, or incomplete spinal cord injury rehabilitation by enhancing neuroplasticity and functional digit movement recovery. The ES device is further intended for, but not limited to, assisting in regaining hand function. The ES device is further intended for, but not limited to, supporting conditions such as multiple sclerosis or muscular dystrophy where hand function is impaired.

The ES device is further intended for, but not limited to, enabling patients to perform essential daily activities, such as gripping and releasing utensils and using tools. The ES device is further intended for, but not limited to, reducing muscle atrophy or spasm.

The ES device is lightweight and designed for daily wear. It can be self-operated by patients without requiring constant professional supervision. It can have adaptive stimulation protocols tailored to individual patient needs to enhance therapeutic outcomes. It can be integrated with digital health platforms to provide data tracking and remote monitoring to improve rehabilitation adherence and effectiveness. It can have manual override mechanisms, such as button or voice control, to ensure continued therapy even in cases of significant motor impairment, and particularly in cases in which patients require extra assistance for movement of their digits.

### Conclusion

This wearable ES device offers an innovative solution for rehabilitation, improving the ability of patients to regain control over the movement of their digits. By combining targeted electrical stimulation with advanced biofeedback mechanisms and manual movement control, the device represents a significant advancement in functional neurorehabilitation technology. The inclusion of manual or voice command control options, adjustable stimulation parameters, and real-time data tracking ensures that the device is both effective and adaptable to a wide range of rehabilitation needs. The invention addresses critical gaps in existing ES solutions by prioritizing usability, functionality, and patient-centered design, making it an essential tool in the recovery of individuals affected by neurological impairments.

1. An electrical stimulation device for assisting movement rehabilitation of digits of a patient, the electrical stimulation device comprising:
a control unit to be worn on a forearm of the patient, the control unit comprising:
electronic circuitry configured to provide electrical pulses;
a first electrode connected to the electronic circuitry to receive electrical signals in the forearm from the patient's brain for triggering the electrical pulses; and
a second electrode connected to the electronic circuitry to receive the electrical pulses and positioned on the forearm such that the electrical pulses stimulate movement of the digits; and
an electrical switch connected to the electronic circuitry and operated by the patient's unaffected hand for triggering the electrical pulses; and
an adjustable band for holding the control unit on the forearm.

2. The electrical stimulation device of claim 1, wherein the electrical switch is a button positioned on the control unit.

3. The electrical stimulation device of claim 1, wherein the electrical switch is a touch-sensitive switch represented by an icon on a touchscreen of the control unit.

4. The electrical stimulation device of claim 1, wherein the electrical switch is a voice-activated unit.

5. The electrical stimulation device of claim 4, wherein the voice-activated unit responds to specific words to stimulate movement of the digits.

6. The electrical stimulation device of claim 1, further comprising wireless connectivity via Bluetooth or Wi-Fi to an external application, enabling customization of therapy sessions and tracking of progress.

7. The electrical stimulation device of claim 1, wherein the electronic circuitry provides the electrical pulses with an adjustable pulse width of 20 to 300 μs, an adjustable frequency of 1 to 100 Hz, and an adjustable amplitude of 1 to 100 mA.

8. The electrical stimulation device of claim 7, wherein the electrical stimulation device is configured to operate in a biofeedback-responsive mode that adjusts the adjustable pulse width, the adjustable frequency, or the adjustable amplitude based on the patient's muscle activation levels detected by the first electrode.

9. The electrical stimulation device of claim 1, wherein the control unit further comprises a rechargeable battery and a charging port.
